(19) United States
(12) Patent Application Publication
(10) Pub. No.: US 2022/0287345 A1
(43) Pub. Date: Sep. 15, 2022

(54) SOLUBILITY ENHANCEMENT OF POORLY SOLUBLE ACTIVES

(71) Applicant: Evonik Operations GmbH, Essen (DE)

(72) Inventors: Ashish GUHA, Mumbai (IN); Sonam SINGH, Thane (IN); Shraddha JOSHI, Thane (IN); Peter NIEPOTH, Gross-Umstadt (DE); Kathrin NOLLENBERGER, Darmstadt (DE); Prajakta SURVE, Thane (W) (IN); Priyanka HAKSAR, Thane (West) (IN)

(73) Assignee: Evonik Operations GmbH, Essen (DE)

(21) Appl. No.: 17/633,533

(22) PCT Filed: Aug. 7, 2020
(86) PCT No.: PCT/EP2020/072232

§ 371 Date: Feb. 7, 2022

(30) Foreign Application Priority Data
Aug. 8, 2019 (IN) 201941032101
Oct. 10, 2019 (EP) 19202434.7

(51) Int. Cl. A23L 33/12; A61K 31/593; A23L 33/15; A61K 31/635; A61K 9/20

(52) U.S. Cl. CPC A23L 33/12; A61K 31/593; A23L 33/15; A61K 31/635; A61K 9/2013; A61K 9/2095

(57) ABSTRACT

The invention provides preparations comprising at least one polyunsaturated fatty acid salt for use in enhancing the solubility in aqueous media for a pharmaceutical or nutraceutical active ingredient in comparison to the pharmaceutical or nutraceutical active ingredient alone by at least 100%, preferably at least 300%. Moreover, a method for preparing a pharmaceutical or nutraceutical dosage form comprising at least one polyunsaturated fatty acid salt and at least one pharmaceutical or nutraceutical active ingredient is disclosed.

The invention provides solubility enhancement of poorly soluble actives with salts of polyunsaturated fatty acids.

Polyunsaturated fatty acids (PUFAs), such as omega-3 fatty acids, particularly eicosapentaenoic acid (EPA) and docosahexaenoic acid (DHA), are linked to numerous positive health effects on the cardiovascular system, on inflammatory disorders, on brain development and function, on disruptions of the central nervous system and on other areas (C. H. S. Ruxton, S. C. Reed, M. J. A. Simpson, K. J. Millington, J. Hum. Nutr. Dietet 2004, 17, 449). Therefore, the intake of omega-3 fatty acids is supported by statements of regulatory agencies. For instance, the EFSA (European Food Safety Authority) recommends for adults a daily intake of 250 mg of EPA+DHA (EFSA Panel on Dietetic Products, Nutrition and Allergies, EFSA Journal 2010, 8 (3), 1461). The AHA (American Heart Association) advises the intake of at least two meals of fatty fish per week for persons without documented cardiovascular disorders, the intake of about 1 g of EPA+DHA per day from fish or food supplements for persons with documented cardiovascular disorders and the intake of 2-4 g of EPA+DHA per day for the treatment of raised blood lipid values (P. M. Kris-Etherton, W. S. Harris, L. J. Appel, Circulation 2002, 106, 2747). Moreover, the authorities have expressly approved health claims for omega-3 fatty acids determined on the basis of clinical studies (EU Register on Nutrition and Health Claims; see also: EFSA Journal 2011, 9 (4), 2078). Therefore, omega-3 fatty acids, especially from fish oil but also from other plant or microbial sources, are increasingly used as food supplements, food additives and medicaments.

According to standard nomenclature, polyunsaturated fatty acids are classified according to the number and position of the double bonds. There are two series or families, depending on the position of the double bond which is closest to the methyl end of the fatty acid. The omega-3 series comprises a double bond at the third carbon atom whereas the omega-6 series has no double bond up to the sixth carbon atom. Thus, docosahexaenoic acid (DHA) has a chain length of 22 carbon atoms with 6 double bonds beginning with the third carbon atom from the methyl end and is referred to as “22:6 n-3” (all-cis-4,7,10,13,16,19-docosahexaenoic acid). Another important omega-3 fatty acid is eicosapentaenoic acid (EPA), which is referred to as “20:5 n-3” (all-cis-5,8,11,14,17-eicosapentaenoic acid).

Most of the omega-3 fatty acid products introduced to the market are offered in the form of oils, starting from fish oil with a content of about 30% omega-3 fatty acids up to concentrates with over 90% content of EPA or DHA or mixtures of these two omega-3 fatty acids. The formulations used are predominantly soft gelatine capsules. In addition, numerous further product forms have been described, such as microencapsulations or powder preparations (C. J. Barrow, B. Wang, B. Adhikari, H. Liu, Spray drying and encapsulation of omega-3 oils, in: Food enrichment with omega-3 fatty acids (Eds.: C. Jacobsen, N. S. Nielsen, A. Frisenfeldt Horn, A.-D. Moltke Soerensen), pp. 194-225, Woodhead Publishing Ltd., Cambridge 2013, ISBN 978-0-85709-428-5; T.-L. Torgersen, J. Klaveness, A. H. Myrset, US 2012/0156296 A1). Chemically, these are usually triglycerides or fatty acid ethyl esters with various concentrations of omega-3 fatty acids, while phospholipids, e.g. as krill oil, free fatty acids (T. J. Maines, B. N. M. Machielse, B. M. Mehta, G. L. Wisler, M. H. Davidson, P. R. Wood, US 2013/0209556 A1; M. H. Davidson, G. H. Wisler, US 2013/0095179 A1; N. J. Duragkar, US 2014/0018558 A1; N. J. Duragkar, US 2014/0051877 A1) and various salts of fatty acids are also known, e.g. with potassium, sodium, ammonium (H. J. Hsu, S. Trusovs, T. Popova, U.S. Pat. No. 8,203,013 B2), calcium and magnesium, (J. A. Kralovec, H. S. Ewart, J. H. D. Wright, L. V. Watson, D. Dennis, C. J. Barrow, J. Functional Foods 2009, 1, 217; G. K. Strohmaier, N. D. Luchini, M. A. Varcho, E. D. Frederiksen, U.S. Pat. No. 7,098,352 B2), where these salts are not water-soluble, aminoalcohols (P. Rongved, J. Klaveness, US 2007/0213298 A1), amine compounds such as piperazine (B. L. Mylari, F. C. Sciavolino, US 2014/0011814 A1), and guanidine compounds such as metformin (M. Manku, J. Rowe, US 2012/0093922 A1; B. L. Mylari, F. C. Sciavolino, US 2012/0178813 A1; B. L. Mylari, F. C. Sciavolino, US 2013/0281535 A1; B. L. Mylari, F. C. Sciavolino, WO 2014/011895 A2). The bioavailability of the different omega-3 derivatives for the human body is very diverse. Since omega-3 fatty acids as free fatty acids together with monoacyl glycerides are absorbed in the small intestine, the bioavailability of free omega-3 fatty acids is better than that of triglycerides or ethyl esters since these have firstly to be cleaved to the free fatty acids in the digestive tract (J. P. Schuchhardt, A. Hahn, Prostaglandins Leukotrienes Essent. Fatty Acids 2013, 89, 1). The stability to oxidation is also very different in different omega-3 derivatives. Free omega-3 fatty acids are described as very sensitive to oxidation (J. P. Schuchhardt, A. Hahn, Prostaglandins Leukotrienes Essent. Fatty Acids 2013, 89, 1). For the use of a solid omega-3 form, an increased stability compared to liquid products is assumed (J. A. Kralovec, H. S. Ewart, J. H. D. Wright, L. V. Watson, D. Dennis, C. J. Barrow, J. Functional Foods 2009, 1, 217).

Furthermore, preparations of omega-3 fatty acids with diverse amino acids, such as lysine and arginine, are known, either as mixtures (P. Literati Nagy, M. Boros, J. Szilbereky, I. Racz, G. Soos, M. Koller, A. Pinter, G. Nemeth, DE 3907649 A1) or as salts (B. L. Mylari, F. C. Sciavolino, WO 2014/011895 A1; T. Bruzzese, EP 0699437 A1; T. Bruzzese, EP0734373 B1; T. Bruzzese, U.S. Pat. No. 5,750,572, J. Torras et al., Nephron 1994, 67, 66; J. Torras et al., Nephron 1995, 69, 318; J. Torras et al., Transplantation Proc. 1992, 24 (6), 2583; S. El Boustani et al., Lipids 1987, 22 (10), 711; H. Shibuya, US 2003/0100610 A1). The preparation of omega-3 aminoalcohol salts by spray-drying is also mentioned (P. Rongved, J. Klaveness, US 2007/0213298 A1).

EP 0734373 B1 describes the preparation of DHA amino acid salts by evaporation to dryness under high vacuum and low temperature or freeze-drying. The resulting products are described as very thick, transparent oils which transform at low temperature into solids of waxy appearance and consistency. Although a tableting formulation has also been mentioned with the use of significant amount of adsorbing diluents, using such oily substance for tableting at larger scales poses significant processing challenges. Moreover, the consistency of such tablets at different temperatures of storage could be altered.

WO 2016/102323 A1 and WO2016/102316 A1 disclose processes for increasing the stability of a composition comprising polyunsaturated omega-3 fatty acids or omega-6 fatty acids against oxidation. The processes comprise the following steps: (i) providing a starting composition comprising at least one polyunsaturated omega-3 or omega-6 fatty acid component; (ii) providing a lysine composition; (iii) admixing aqueous, aqueous-alcoholic or alcoholic solutions of starting composition and lysine composition, and subjecting resulting admixture to spray drying conditions subsequently, thus forming a solid product composition comprising at least one salt of a cation derived from lysine with an anion derived from a polyunsaturated omega-3 or omega-6 fatty acid. Although in this invention a useful process for production of solid PUFA salt of amino acid is described using spray drying conditions, the powder obtained at the end lacks useful properties necessary for production of dosage forms like tablets.

Solubility is one of the important parameters to attain desired concentration of drugs in systemic circulation for pharmacological response to be shown. Solubility is defined in quantitative terms as the concentration of solute in a saturated solution at a certain temperature and in a qualitative way, it may be defined as the spontaneous interaction of two or more substances to form a homogenous molecular dispersion (Physical Pharmacy: Alfred Martin). The maximum equilibrium amount of solute that can dissolve per amount of solvent is the solubility of that solute in that solvent under the specified conditions (commonly expressed as a concentration).

The Biopharmaceutics Classification System is a system to differentiate the drugs based on their solubility and permeability. The solubility classification is based on a United States Pharmacopoeia (USP) aperture. According to the Biopharmaceutical Classification System (BCS) drug substances are classified to four classes upon their solubility and permeability:
Class I—high permeability, high solubility: Those compounds are well absorbed, and their absorption rate is usually higher than excretion.
Class II—high permeability, low solubility: The bioavailability of those products is limited by their solvation rate. A correlation between the in vivo bioavailability and the in vitro solvation can be found.
Class III—low permeability, high solubility: The absorption is limited by the permeation rate, but the drug is solvated very fast. If the formulation does not change the permeability or gastro-intestinal duration time, then class I criteria can be applied.
Class IV—low permeability, low solubility: Those compounds have a poor bioavailability. Usually they are not well absorbed over the intestinal mucosa and a high variability is expected.

It is vital to improve the solubility and dissolution rate for poorly soluble drugs (especially for substances classified as BCS classes II, III or IV), since these drugs possess low absorption and bioavailability. About 40% of all new chemical entities have poor bioavailability. Increasing the bioavailability of poorly soluble drugs is one big challenge for formulation scientists. A review article highlights all the methods used for the solubility enhancement along with the polymers which are used as carrier for increasing the solubility of the poorly soluble drugs. Polymers which can be used as a carrier belong to categories like water soluble polymers (povidone (PVP)), polyethylene glycol (PEG), cyclodextrin, hydroxypropyl methyl cellulose, methyl cellulose, hydroxy ethyl cellulose, hydroxy propyl cellulose, acid (citric acid, succinic acid), hydrotops (urea, sodium acetate, nicotinamide, sodium benzoate, sodium salicylate, sodium-hydroxy benzoate), sugars (dextrose, sucrose, galactose, sorbitol, maltose, mannitol, lactose), surfactants (deoxycholic acid, tweens, spans, polyoxyethylene stearate, renex, poloxamer 188.), insoluble or enteric polymers (EUDRAGIT® L 100, EUDRAGIT® S 100, EUDRAGIT® RL, EUDRAGIT® RS, hydroxy propyl methyl cellulose phthalate). None of the techniques and carrier systems is optimal as they have their inherent drawbacks (Gaikwad et al., Review on: Solubility Enhancement of Poorly Water Soluble Drug. Indo American Journal of Pharm Research. 2014:4-11).

In a specific study, solubility enhancement of Nimesulide was targeted by using Mannitol and EUDRAGIT® E PO by milling technique. With the dissolution studies which were carried out in water it was observed that Mannitol was able to give more release compared to EUDRAGIT® EPO and this was mainly because Mannitol being hydrophilic in nature helped in increasing the dissolution profile of the drug in all the dosage form, whereas EUDRAGIT® E PO showed lesser release which is because of the improper miscibility of EUDRAGIT® E PO in aqueous media due to its hydrophobicity and its solubility in acidic pH. Thus, pH dependent solubility is prominent with EUDRAGIT® E PO (Dantu et al., Enhancement of Solubility of Nimesulide in the Presence of Polymer with Milling Technique, /J. Pharm. Sci. & Res. Vol. 4(9), 2012, 1907-1914).

Another study aimed to determine the better carrier for the solubility enhancement of Ritonavir. Two carriers were selected, i.e. Kolliphor® P188 and EUDRAGIT® L 100-55. Dissolution was carried out in pH 1.2 in 0.1 N HCl followed by pH 6.8 and even in FaSSIF and FeSSIF conditions. Kolliphor® P188 is a water-soluble excipient, so it showed a pH-independent release profile, whereas EUDRAGIT® L100-55 being a pH-dependent polymer showed release profile of the drug same as that in the pure form and then complete release in pH 6.8 as EUDRAGIT® L100-55 releases above 5.5 pH. Same observation was seen with FaSSIF and FeSSIF condition, in FaSSIF pH being more than pH 6, complete release of the drug was obtained with EUDRAGIT® L100-55 but in FeSSIF condition slower release of the drug was observed due to the acidic condition of the media (Dhore et al., Influence of carrier (polymer) type and drug carrier ratio in the development of amorphous dispersions for solubility and permeability enhancement of ritonavir. St. John Fisher College Fisher Digital Publications, 9-23-2017).

### Problem

Poor solubility of a huge number of active pharmaceutical and nutraceutical ingredients including the new chemical entities, is one of the major formulation challenges faced by the formulators. Such molecules, despite having high therapeutic effectiveness, find limited potential use in formulating bioavailable products. In these cases, the rate limiting factor for absorption of actives is dissolution rate in gastrointestinal media. There is a need of more formulation and technology solutions in the field of new formulations development.

Ionic excipients like some of the methacrylate polymers (such as EUDRAGUARD® protect and EUDRAGIT® L100-55), do offer good solubility increase for the counter ionic drugs but suffer from disadvantages such as pH specific solubility increase with little or no solubility in the opposite pH conditions limiting their use in formulations.

### Solution

It was found that the salts of polyunsaturated fatty acids are not just healthy food supplements, but when combined with poorly soluble advanced food ingredients (AFI) and active pharmaceutical ingredients (API), via specific processes, can increase the solubility of actives in the aqueous media, which could help in increasing bioavailability and efficacy of such active ingredients. Further to this, surprisingly, it was also found that such amino acid salts (and magnesium and potassium salts) of polyunsaturated fatty acids exert synergistic increase in solubility of actives when combined with ionic methacrylate polymers. Furthermore, interestingly, the pH dependency of such formulations is also minimized with such synergistic combinations.

The invention offers an alternate solution to the low solubility problems of a large number of active pharmaceutical and nutraceutical ingredients. The proposed synergistic combination may help in achieving better bioavailability and efficacy with potentially low doses of such active ingredients.

In the context of the present invention the term PUFA is used interchangeably with the term polyunsaturated fatty acid and defined as follows: Fatty acids are classified based on the length and saturation characteristics of the carbon chain. Short chain fatty acids have 2 to about 6 carbons and are typically saturated. Medium chain fatty acids have from about 6 to about 14 carbons and are also typically saturated. Long chain fatty acids have from 16 to 24 or more carbons and may be saturated or unsaturated. In longer chain fatty acids there may be one or more points of unsaturation, giving rise to the terms “monounsaturated” and “polyunsaturated,” respectively. In the context of the present invention long chain polyunsaturated fatty acids having 20 or more carbon atoms are designated as polyunsaturated fatty acids or PUFAs.

PUFAs are categorized according to the number and position of double bonds in the fatty acids according to well established nomenclature. There are two main series or families of LC-PUFAs, depending on the position of the double bond closest to the methyl end of the fatty acid: The omega-3 series contains a double bond at the third carbon, while the omega-6 series has no double bond until the sixth carbon. Thus, docosahexaenoic acid (“DHA”) has a chain length of 22 carbons with 6 double bonds beginning with the third carbon from the methyl end and is designated “22:6 n-3” (all-cis-4,7,10,13,16,19-docosahexaenoic acid). Another important omega-3 PUFA is eicosapentaenoic acid (“EPA”) which is designated “20:5 n-3” (all-cis-5,8,11,14,17-eicosapentaenoic acid). An important omega-6 PUFA is arachidonic acid (“ARA”) which is designated “20:4 n-6” (all-cis-5,8,11,14-eicosatetraenoic acid).

Other omega-3 PUFAs include: Eicosatrienoic acid (ETE) 20:3 (n-3) (all-cis-11,14,17-eicosatrienoic acid), Eicosatetraenoic acid (ETA) 20:4 (n-3) (all-cis-8,11,14,17-eicosatetraenoic acid), Heneicosapentaenoic acid (HPA) 21:5 (n-3) (all-cis-6,9,12,15,18-heneicosapentaenoic acid), Docosapentaenoic acid (Clupanodonic acid) (DPA) 22:5 (n-3) (all-cis-7,10,13,16,19-docosapentaenoic acid), Tetracosapentaenoic acid 24:5 (n-3) (all-cis-9,12,15,18,21-tetracosapentaenoic acid), Tetracosahexaenoic acid (Nisinic acid) 24:6 (n-3) (all-cis-6,9,12,15,18,21-tetracosahexaenoic acid).

Other omega-6 PUFAs include: Eicosadienoic acid 20:2 (n-6) (all-cis-11,14-eicosadienoic acid), Dihomo-gamma-linolenic acid (DGLA) 20:3 (n-6) (all-cis-8,11,14-eicosatrienoic acid), Docosadienoic acid 22:2 (n-6) (all-cis-13,16-docosadienoic acid), Adrenic acid 22:4 (n-6) (all-cis-7,10,13,16-docosatetraenoic acid), Docosapentaenoic acid (Osbond acid) 22:5 (n-6) (all-cis-4,7,10,13,16-docosapentaenoic acid), Tetracosatetraenoic acid 24:4 (n-6) (all-cis-9,12,15,18-tetracosatetraenoic acid), Tetracosapentaenoic acid 24:5 (n-6) (all-cis-6,9,12,15,18-tetracosapentaenoic acid).

Preferred omega-3 PUFAs used in the embodiments of the present invention are docosahexaenoic acid (DHA) and eicosapentaenoic acid (EPA).

Compositions comprising polyunsaturated omega-3 or omega-6 fatty acids that can be used for the process of the present invention may be any compositions containing substantial amounts of free polyunsaturated omega-3 or omega-6 fatty acids. Such compositions may further comprise other naturally occurring fatty acids in free form. In addition, such compositions may further comprise constituents that by themselves are solid, liquid or gaseous at room temperature and standard atmospheric pressure. Corresponding liquid constituents include constituents that can easily be removed by evaporation and could thus be considered as volatile constituents as well as constituents that are difficult to remove by evaporation and could thus be considered as non-volatile constituents. In the present context gaseous constituents are considered as volatile constituents. Typical volatile constituents are water, alcohols and supercritical carbon dioxide.

Compositions comprising polyunsaturated omega-3 fatty acids that can be used for the process of the present invention may be obtained from any suitable source material which, additionally, may have been processed by any suitable method of processing such source material. Typical source materials include any part of fish carcass, vegetables and other plants as well as material derived from microbial and/or algal fermentation. Typically, such material further contains substantial amounts of other naturally occurring fatty acids. Typical methods of processing such source materials may include steps for obtaining crude oils such as extraction and separation of the source material, as well as steps for refining crude oils such as settling and degumming, de-acidification, bleaching, and deodorization, and further steps for producing omega-3 omega-6 PUFA-concentrates from refined oils such as de-acidification, trans-esterification, concentration, and deodorization (cf. e.g. EFSA Scientific Opinion on Fish oil for Human Consumption). Any processing of source materials may further include steps for at least partially transforming omega-3 omega-6 PUFA-esters into the corresponding free omega-3 PUFAs or inorganic salts thereof.

Preferred compositions comprising polyunsaturated omega-3 fatty acids used for the process of the present invention can be obtained from compositions mainly consisting of esters of omega-3 PUFAs and other naturally occurring fatty acids by cleavage of the ester bonds and subsequent removal of the alcohols previously bound as esters. Preferably, ester cleavage is performed under basic conditions. Methods for ester cleavage are well known in the art.

The present invention is directed to the use of a preparation comprising at least one polyunsaturated fatty acid salt comprising at least one omega-3 fatty acid selected from eicosapentaenoic acid (EPA) and docosahexaenoic acid (DHA) for enhancing the solubility in aqueous media for a pharmaceutical or nutraceutical active ingredient in comparison to the pharmaceutical or nutraceutical active ingredient alone by at least 100%, preferably at least 300%.

Salts of polyunsaturated fatty acids when combined with poorly soluble APIs and AFIs, can increase the solubility of actives in the aqueous media, which could help in increasing bioavailability and efficacy of such active ingredients.

In a preferred configuration, the pharmaceutical active ingredient is selected from BCS classes II, III or IV. Moreover, it is preferred, when the pharmaceutical active ingredient is ionic.

The omega-3 fatty acid preferably comprises both EPA and DHA. In a preferred embodiment, the omega-3 fatty acid salts have an organic counter ion selected from lysine, arginine, ornithine, choline or at least counter ion selected from magnesium (Mg²⁺) and potassium (K⁺) and mixtures of the same.

It is particularly preferred, when the omega-3 fatty acid salt comprises as counter ion lysine or a mixture of lysine and one or more of arginine, ornithine, magnesium and potassium, wherein the ratio between lysine and arginine, ornithine, magnesium and potassium is between 10:1 and 1:1.

In a specific configuration it is preferred to use a omega-3 fatty acid salt comprising as counter ion mixtures of lysine and arginine, lysine and ornithine, arginine and ornithine, magnesium and lysine or potassium and lysine.

In the context of the present invention a cation derived from a basic amine selected from lysine, arginine, ornithine, choline, or mixtures thereof is a cation obtained by protonation of lysine, arginine, ornithine, choline, or mixtures thereof.

In the context of the present invention an anion derived from a polyunsaturated omega-3 fatty acid is an anion obtained by deprotonation of a polyunsaturated omega-3 fatty acid.

In a further preferred configuration, the source for omega-3 fatty acids is chosen from at least one of the following: fish oil, squid oil, krill oil, linseed oil, borage seed oil, algal oil, hemp seed oil, rapeseed oil, flaxseed oil, canola oil, soybean oil.

In a preferred embodiment, the amount of polyunsaturated fatty acid is 65 weight % or less, preferably 60 weight % or less, more preferably between 40 and 55 weight-% with respect to the total weight of polyunsaturated fatty acid salt.

It was surprisingly found that such amino acid salts of polyunsaturated fatty acids exert synergistic increase in solubility of actives when combined with ionic polymers. Furthermore, interestingly, the pH dependency of such formulations is also minimized with such synergistic combinations.

Therefore, in a preferred embodiment, the preparation further comprises an ionic polymer, preferably selected from cationic (meth)acrylate copolymers, anionic (meth)acrylate copolymers, anionic cellulose derivatives, alginates and polyacrylic acid.

The ionic polymer may be a cationic copolymer, such as an “amino methacrylate copolymer (USP/NF)”, “basic butylated methacrylate copolymer (Ph. Eur)” or “aminoalkyl methacrylate Copolymer E (JPE)” which are of the EUDRAGIT® E type. Suitable EUDRAGIT® E type copolymers are known, for example, from EP 0 058 765 B1.

The amino (meth)acrylate copolymer may be composed, for example, of 30 to 80% by weight of free-radically polymerized C1- to C4-alkyl esters of acrylic acid or of methacrylic acid, and 70 to 20% by weight of (meth)acrylate monomers having a tertiary amino group in the alkyl radical.

Suitable monomers with functional tertiary amino groups are detailed in U.S. Pat. No. 4,705,695, column 3 line 64 to column 4 line 13. Mention should be made in particular of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate, diethylamino-2,2-dimethyl)propyl methacrylate and diethylaminoethyl methacrylate. Particular preference is given to dimethylaminoethyl methacrylate.

The content of the monomers with tertiary amino groups in the copolymer may advantageously be between 20 and 70% by weight, preferably between 40 and 60% by weight. The proportion of the C1- to C4-alkyl esters of acrylic acid or methacrylic acid is 70-30% by weight. Mention should be made of methyl methacrylate, ethyl methacrylate, butyl methacrylate, methyl acrylate, ethyl acrylate and butyl acrylate.

A suitable amino (meth)acrylate copolymer may be polymerized out of, for example, from 20-30% by weight of methyl methacrylate, 20-30% by weight of butyl methacrylate and 60-40% by weight of dimethylaminoethyl methacrylate.

A specifically suitable commercial amino (meth)acrylate copolymer is, for example, formed from 25% by weight of methyl methacrylate, 25% by weight of butyl methacrylate and 50% by weight of dimethylaminoethyl methacrylate (EUDRAGIT® E 100 or EUDRAGIT® E PO (powder form)). EUDRAGIT® E 100 and EUDRAGIT® E PO are water-soluble below approx. pH 5.0 and are thus also gastric juice-soluble.

The ionic polymer may also be an anionic (meth)acrylate copolymer, which may comprise 25 to 95, preferably 40 to 95, in particular 40 to 60% by weight of free-radical polymerized C1- to C4-alkyl esters of acrylic or of methacrylic acid and 75 to 5, preferably 60 to 5, in particular 60 to 40% by weight of (meth)acrylate monomers having an anionic group.

The proportions mentioned normally add up to 100% by weight. However, it is also possible in addition, without this leading to an impairment or alteration of the essential properties, for small amounts in the region of 0 to 10, for example 1 to 5, % by weight of further monomers capable of vinylic copolymerization, such as, for example, hydroxyethyl methacrylate or hydroxyethyl acrylate, to be present. It is preferred that no further monomers capable of vinylic copolymerization are present.

C1- to C4-alkyl esters of acrylic or methacrylic acid are in particular methyl methacrylate, ethyl meth-acrylate, butyl methacrylate, methyl acrylate, ethyl acrylate and butyl acrylate.

A (meth)acrylate monomer having an anionic group is, for example, acrylic acid, with preference for methacrylic acid.

Suitable anionic (meth)acrylate copolymers are those composed of 40 to 60% by weight methacrylic acid and 60 to 40% by weight methyl methacrylate or 60 to 40% by weight ethyl acrylate (EUDRAGIT® L 100 or EUDRAGIT® L 100-55 types).

EUDRAGIT® L 100 is a copolymer of 50% by weight methyl methacrylate and 50% by weight methacrylic acid. The pH of the start of the specific active ingredient release in intestinal juice or simulated intestinal fluid can be stated to be pH 6.0.

EUDRAGIT® L 100-55 is a copolymer of 50% by weight ethyl acrylate and 50% by weight methacrylic acid. EUDRAGIT® L 30 D-55 is a dispersion comprising 30% by weight EUDRAGIT® L 100-55. The pH of the start of the specific active ingredient release in intestinal juice or simulated intestinal fluid can be stated to be pH 5.5.

Likewise, suitable are anionic (meth)acrylate copolymers composed of 20 to 40% by weight methacrylic acid and 80 to 60% by weight methyl methacrylate (EUDRAGIT® S type). The pH of the start of the specific active ingredient release in intestinal juice or simulated intestinal fluid can be stated to be pH 7.0.

Suitable (meth)acrylate copolymers are those consisting of 10 to 30% by weight methyl methacrylate, 50 to 70% by weight methyl acrylate and 5 to 15% by weight methacrylic acid (EUDRAGIT® FS type). The pH at the start of the specific active ingredient release in intestinal juice or simulated intestinal fluid can be stated to be pH 7.0.

The (meth)acrylate copolymer comprises 10% to 30%, preferably 15% to 30%, more preferably 20% to 30% by weight of methyl methacrylate units, 50% to 70%, preferably 55% to 70%, more preferably 60% to 70%, by weight of methyl acrylate units, 5% to 15%, preferably 6% to 14%, more preferably, 8% to 12%, by weight of methacrylic acid units.

Especially preferred for the use in the composition according to the invention is a copolymer containing 25% by weight methyl methacrylate, 65% by weight methyl acrylate and 10% by weight methacrylic acid (EUDRAGIT® FS manufactured by Evonik Industries).

The anionic (meth)acrylate copolymer may also be composed of free radical polymerized methyl methacrylate, ethylacrylate and a salt of 2-trimethylammoniumethyl methacrylate. These kinds of copolymers may be used for sustained release coating compositions or sustained release matrix compositions.

The (meth)acrylate copolymer may be composed of 85-98% by weight of methyl methacrylate and ethyl acrylate and 15 to 2% by weight of a salt of 2-trimethylammoniumethyl methacrylate, preferably, 2-trimethylammoniumethyl methacrylate chloride. The weight percentages add up to 100%.

The (meth)acrylate copolymer may be composed 50 to 70% by weight of methyl methacrylate, 20 to 40% by weight of ethyl acrylate and 7 to 2% by weight of a salt of 2-trimethylammoniumethyl methacrylate, preferably 2-trimethylammoniumethyl methacrylate chloride (EUDRAGIT® RS type), wherein the weight percentages add up to 100%.

A specifically suitable copolymer comprises 65% by weight of methyl methacrylate, 30% by weight of ethyl acrylate and 5% by weight of 2-trimethylammoniumethyl methacrylate chloride (EUDRAGIT® RS).

Further, the (meth)acrylate copolymer may be composed of 50 to 70% by weight of methyl methacrylate, 20 to 40% by weight of ethyl acrylate and more than 7 up to 12% by weight of a salt of 2-trimethylammoniumethyl methacrylate, preferably 2-trimethylammoniumethyl methacrylate chloride (EUDRAGIT® RL type), wherein the weight percentages add up to 100%.

A specifically suitable copolymer comprises 65% by weight of methyl methacrylate, 30% by weight of ethyl acrylate and 10% by weight of 2-trimethylammoniumethyl methacrylate chloride (EUDRAGIT® RL).

A further subject of the present invention is a method of preparing a dosage form comprising at least one polyunsaturated fatty acid salt comprising at least one omega-3 fatty acid selected from EPA and DHA and at least one pharmaceutical or nutraceutical active ingredient, comprising the steps of:

a. co-processing of at least one polyunsaturated fatty acid salt comprising at least one omega-3 fatty acid selected from EPA and DHA and one pharmaceutical or nutraceutical active ingredient and optionally pharmaceutically or nutraceutically acceptable excipients;

b. optionally mixing of the co-processed components from step a. with one or more excipients; and

c. formulating the components to produce a dosage form.

In a preferred embodiment, the dosage form is a tablet and the mixed components from step b. are compressed in a tableting machine to produce a tablet and the ejection force experienced by the tableting machine is not more than 150N.

It is particularly preferred, when the ratio of the active ingredient to polyunsaturated fatty acid salt is between 1:0.5 to 1:50.

In an advantageous configuration of the present invention, the polyunsaturated fatty acid salt and the pharmaceutical or nutraceutical active ingredient are co-processed with one or more ionic polymers. It is preferred, when the ionic polymer is selected from cationic (meth)acrylate copolymers, anionic (meth)acrylate copolymers, anionic cellulose derivatives, alginates and polyacrylic acid.

In a preferred embodiment, the ratio of the active ingredient to the ionic polymer is between 0.1:1 to 1:0.1. Moreover, it is preferred, when the ratio of polyunsaturated fatty acid salt and the ionic polymer is between 0.2:1 to 1:0.2.

In a further preferred embodiment, the co-processing in step a. comprises one or more of the following: spray drying conditions selected from pure spray drying and spray-agglomeration or co-milling, freeze drying, physical mixing, co-sifting, vacuum drying, hot-melt extrusion, compaction, slugging, 3D printing, molding, film casting and coating.

It is particularly preferred to use spray drying conditions selected from pure spray drying and spray-agglomeration or physical mixing.

It is preferred, when prior to step a. the pharmaceutical or nutraceutical active ingredient is mixed with one or more lipophilic substances. It is further preferred, when the lipophilic substance is capable of dissolving at least 300% of the amount of pharmaceutical or nutraceutical active ingredient as compared to water.

In a preferred configuration, the pharmaceutical active ingredient is selected from BCS classes II, III or IV. Moreover, it is preferred, when the pharmaceutical active ingredient is ionic.

A further subject of the present invention is a pharmaceutical or nutraceutical dosage form prepared according to the present invention.

In the context of the present invention nutraceutical dosage forms comprise any type of nutraceutical, nutrient or dietary supplement, e.g. for supplementing vitamins, minerals, fiber, fatty acids, or amino acids.

The pharmaceutical or nutraceutical dosage form is preferably selected from tablets, capsules, soft capsules, suspensions, emulsions, granules, powders, oral films, pellets, suppositories, pessaries, intra-vascular dosage forms.

The pharmaceutical or nutraceutical dosage form may further comprise excipients, wherein the excipients are selected from the group of binders, antioxidants, glidants, lubricants, pigments, plasticizers, polymers, brighteners, diluents, flavors, surfactants, pore formers, stabilizers.

In the context of the present invention the pharmaceutical product can further comprise a pharmaceutically acceptable excipient as well as further pharmaceutically active agents including for example cholesterol-lowering agents such as statins, anti-hypertensive agents, anti-diabetic agents, anti-dementia agents, anti-depressants, anti-obesity agents, appetite suppressants and agents to enhance memory and/or cognitive function.

In a preferred configuration, the pharmaceutical active ingredient is selected from BCS classes II, III or IV. Moreover, it is preferred, when the pharmaceutical active ingredient is ionic.

In a preferred configuration, the omega-3 fatty acid component comprises EPA and DHA.

It is preferred when in the pharmaceutical or nutraceutical dosage form, the polyunsaturated fatty acid salt comprises at least one basic amino acid or at least counter ion selected from magnesium (Mg²⁺) and potassium (K⁺). In a further preferred configuration, the omega-3 fatty acid salts have an organic counter ion selected from lysine, arginine, ornithine, choline and mixtures of the same.

The basic amino acids are preferably selected from lysine, arginine, ornithine and mixtures of the same.

In a preferred embodiment, the amount of polyunsaturated fatty acid is 65 weight % or less, preferably 60 weight % or less, more preferably between 40 and 55 weight-% with respect to the total weight of polyunsaturated fatty acid salt.

### EXAMPLES

Polyunsaturated Fatty Acid Compositions

In the examples for the present invention, different polyunsaturated fatty acid compositions were used. Different omega-3 fatty acid salts having an organic counter ion selected from the basic amino acids lysine, arginine and ornithine were prepared. The omega-3 fatty acids Eicosapentaenoic acid (C20:5w3c) (EPA) and Docosahexaenoic acid (C22:6w3c) (DHA) are present in a ratio of around 2:1 (ratio EPA:DHA). The salts were prepared by spray granulation as described in WO2016102323A1.

The omega-3 lysine salt (omega-3-lys) contains around 32 weight-% of L-lysine and around 65 weight-% of polyunsaturated fatty acids (AvailOm®, Evonik Nutrition and Care GmbH, Darmstadt, Germany). The major polyunsaturated fatty acids in the composition are the omega-3 fatty acids Eicosapentaenoic acid (C20:5w3c) (EPA) and Docosahexaenoic acid (C22:6w3c) (DHA), summing up to around 58 weight-% of the composition. The composition also contains minor amounts of Docosaenoic acid isomer (incl. erucic acid) (C22:1), Docosapentaenoic acid (C22:5w3c) and of the omega-6 fatty acids Arachidonic acid (C20:4w6) and Docosatetraenoic acid (C22:4w6c).

The omega-3 arginine salt (omega-3-arg) contains around 35 weight-% of L-arginine and around 64 weight-% of polyunsaturated fatty acids. The major polyunsaturated fatty acids in the composition are the omega-3 fatty acids Eicosapentaenoic acid (C20:5w3c) (EPA) and Docosahexaenoic acid (C22:6w3c) (DHA), summing up to around 49 weight-% of the composition. The composition also contains minor amounts of Docosaenoic acid isomer (incl. erucic acid) (C22:1), Docosapentaenoic acid (C22:5w3c) and of the omega-6 fatty acids Arachidonic acid (C20:4w6) and Docosatetraenoic acid (C22:4w6c).

The omega-3 ornithine salt (omega-3-orn) contains around 29 weight-% of L-ornithine and around 70 weight-% of polyunsaturated fatty acids. The major polyunsaturated fatty acids in the composition are the omega-3 fatty acids Eicosapentaenoic acid (C20:5w3c) (EPA) and Docosahexaenoic acid (C22:6w3c) (DHA), summing up to around 54 weight-% of the composition. The composition also contains minor amounts of Docosaenoic acid isomer (incl. erucic acid) (C22:1), Docosapentaenoic acid (C22:5w3c) and of the omega-6 fatty acids Arachidonic acid (C20:4w6) and Docosatetraenoic acid (C22:4w6c).

The mixed salts of ornithine and arginine (50:50), ornithine and lysine (50:50) and mixed salts of arginine and lysine (50:50) were prepared by spray granulation as described in WO2016102323A1 using the PUFA composition described above. The Mg²⁺ salts and mixed salts of Mg²⁺ and arginine (50:50) were prepared by kneading as described in WO2017202935A1 using the PUFA composition described above.

### Comparative Examples 1-7

Acrylic polymers are widely explored and known for its solubility enhancement effect. As comparative examples (table 1), the solubility enhancement effect of such polymers of the EUDRAGIT® type on different advanced food ingredients (AFI) and active pharmaceutical ingredients (API) in water was evaluated.

TABLE 1
Formulation for comparative experiments,

| Experiment | C-1 | C-2 | C-3 | C-4 | C-5 | C-6 | C-7 |
|---|---|---|---|---|---|---|---|
| Method of Coprocessing | .. | .. | SD | .. | SD | .. | SD |
| End Dosage | Blend | Blend | Blend | Blend | Blend | Blend | Blend |
| form | | | | | | | |
| Ingredient | % w/w | % w/w | % w/w | % w/w | % w/w | % w/w | % w/w |
| Celecoxib | 100 | .. | .. | .. | .. | .. | .. |
| Ritonavir | .. | 100 | 25 | .. | .. | .. | .. |
| Curcumin | .. | .. | .. | 100 | 25 | .. | .. |
| Quercetin | .. | .. | .. | .. | .. | 100 | 14.49 |
| EUDRAGIT ® | .. | .. | 75* | .. | 75* | .. | .. |
| EPO | | | | | | | |
| EUDRAGIT ® | .. | .. | .. | .. | .. | 43.47* | |
| L100-55 | | | | | | | |
| MCC PH 102 | .. | .. | .. | .. | .. | .. | 33.33** |
| Lactose | .. | .. | .. | .. | .. | .. | |
| Total | 100 | 100 | 100 | 100 | 100 | 100 | 100 |
| Ethanol: | .. | .. | 100:0 | .. | 50:50 | .. | 100:0 |
| Methanol | | | | | | | |
| Ratio | | | | | | | |
| Solid Content | .. | .. | 13.55 | .. | 4 | .. | 10 |
| % w/w | | | | | | | |

**MCC PH 102 was added externally

For the examples without coprocessing applied for C-1, C-2, C-4 and C-6, the AFI/API was directly used for analysis. For the spray drying process (SD) applied for C-3, C-5 and C-7, the AFI/API was mixed in the solvent and to this solution EUDRAGIT® was added under constant stirring. The solution was spray dried after a clear solution was obtained (parameters shown in table 2). The spray dried powder obtained was then taken for physical mixing with other tableting excipients if any. The blend was then taken directly for analysis.

TABLE 2
Process parameter for spray drying

| Parameters | C-3 | C-5 | C-7 |
|---|---|---|---|
| Inlet Temperature (° C.) | 42-43 | 43-45 | 43-45 |
| Aspirator (%) | 70-89 | 87-100 | 92-93 |

Analysis of the blend was done and its solubility in water was analyzed (table 3).

TABLE 3
Solubility of AFI/API in water

| Experiment | C-1 | C-2 | C-3 | C-4 | C-5 | C-6 | C-7 |
|---|---|---|---|---|---|---|---|
| Solubility of | 0.1 μg/ml | 0.0 μg/ml | 0.0 μg/ml | 0.0 μg/ml | 0.1 μg/ml | 3.6 μg/ml | 0.1 μg/ml |
| AFI/API in | | | | | | | |
| water | | | | | | | |

For the following experiments, acceptance criteria were defined whether there is any enhancement in solubility of the AFI/API of more than 3 times increase water solubility of the AFI/API in relation to observed solubility in the comparative examples as shown above.

### Examples 1-7: Solubility Enhancement of Food Ingredients Curcumin and Quercetin (Inventive)

TABLE 4
Formulation for food ingredients Curcumin and Quercetin,

| Experiment | I-1 | I-2 | I-3 | I-4 | I-5 | I-6 | I-7 |
|---|---|---|---|---|---|---|---|
| Method of | SD | SD | SD + PM | SD | SD | SD + PM | SD |
| Coprocessing | | | | | | | |
| End Dosage | Blend | Tablet | Tablet | Tablet | Tablet | Tablet | Tablet |
| form | | | | | | | |
| Ingredient | % w/w | % w/w | % w/w | % w/w | % w/w | % w/w | % w/w |
| Curcumin | 9.1* | 9.1* | 15.63* | 10.21* | 6.9* | .. | .. |
| Quercetin | .. | .. | .. | .. | .. | 12.12* | 6.9 |
| MCT Oil | .. | .. | .. | .. | .. | .. | .. |
| AvailOm ® | 90.9* | 90.9* | 37.5** | 27.27* | 31.03* | 18.16** | 31.03 |
| EUDRAGIT ® | .. | .. | 46.9* | 54.55* | 62.07* | .. | 62.1 |
| EPO | | | | | | | |
| EUDRAGIT ® | .. | .. | .. | .. | .. | 36.36* | |
| L100-55 | | | | | | | |
| MCC PH 102 | .. | .. | .. | .. | .. | 33.33 | |
| Total | 100 | 100 | 100 | 100 | 100 | 100 | 100 |
| Ethanol: | 90:10 | 90:10 | .. | 80:20 | 80:20 | 100:0 | 80:20 |
| Water Ratio | | | | | | | |
| Ethanol: | .. | .. | 50:50 | .. | .. | .. | .. |
| Methanol | | | | | | | |
| Ratio | | | | | | | |
| Solid Content | 7.28 | 7.28 | 4 | 9.9 | 12.66 | 10 | 10.39 |
| (% w/w) | | | | | | | |

*ingredients were co-processed together in a single spray drying step,
**ingredients were co-processed by only physically mixing with the spray dried powder in the same batch
Formulations for the food ingredients Curcumin and Quercetin are shown in table 4.

For the spray drying process (SD) applied for I-1, I-2, I-4, I-5 and I-7, the food ingredient was added in the non-aqueous media and to this solution EUDRAGIT® if any was added and then AvailOm® (omega-3 lysine salt) was added under constant stirring. After addition of AvailOm®, the remaining amount of water was added and after the solution getting completely clear used for spray drying (parameters shown in table 5). The spray dried powder obtained was then mixed with other tableting excipients if any by passing through a 30# sieve in geometric addition. The blend was then taken for compression (tableting parameters shown in table 6). For I-1, the blend was directly used for analysis.

TABLE 5
Process parameter for spray drying

| Parameters | I-1 | I-2 | I-4 | I-5 | I-7 |
|---|---|---|---|---|---|
| Inlet Temperature (° C.) | 55-57 | 55-57 | 65-69 | 69-70 | 69-74 |
| Aspirator (%) | 91-100 | 91-100 | 70-89 | 90-100 | 81-100 |

TABLE 6
Tableting parameters

| Parameter | I-2 | I-4 | I-5 | I-7 |
|---|---|---|---|---|
| Punch size (mm) | 12.5 | 10 | 18*8 | 18*8 |
| Average Weight (mg) | 550 | 270-280 | 720-730 | 720-730 |
| Compression force (KN) | 15.23 | 2.948 | 5.848 | 3.672 |
| Ejection force (N) | 104.5 | 96.80 | 96.80 | 98 |
| Hardness (N) | 70-90 | 70-90 | 60-80 | — |

For the spray drying physical mixing process (SD+PM) applied for I-3 and I-6, the food ingredient was added in the solvent, EUDRAGIT® was added to this solution under constant stirring and after the solution getting completely clear used for spray drying (table 7). The spray dried powder obtained was then taken for physical mixing with AvailOm® and other tableting excipients if any. The blend was then taken for compression (tableting parameters shown in table 8).

TABLE 7
Process parameter for spray drying

| Parameters | I-3 | I-6 |
|---|---|---|
| Inlet Temperature (° C.) | 42-43 | 43-45 |
| Aspirator (%) | 87-100 | 89-93 |

TABLE 8
Tableting parameters

| Parameter | I-3 | I-6 |
|---|---|---|
| Punch size (mm) | 11 | 12.5 |
| Average Weight (mg) | 320 | 413 |
| Compression force (KN) | 3.112 | 2.529 |
| Ejection force (N) | 107.30 | 107.140 |
| Hardness (N) | 60-80 | 60-80 |

All the tablets prepared as shown in table 8 were analyzed in water to check whether there is an enhancement in solubility of the drug when compared to the pure form (table 9).

TABLE 9
Solubility of AFI,

| Experiment | I-1 | I-2 | I-3 | I-4 | I-5 | I-6 | I-7 |
|---|---|---|---|---|---|---|---|
| Solubility of | 11 μg/ml | 11 μg/ml | 3 μg/ml | 60 μg/ml | 107 μg/ml | 33 μg/ml | 17.9 μg/ml |
| AFI in water | | | | | | | |
| % Increase in | * >300% | * >300% | * >300% | * >300% | * >300% | 917 | 497 |
| Solubility | | | | | | | |
| Acceptance | Yes | Yes | Yes | Yes | Yes | Yes | Yes |
| criteria met | | | | | | | |

* The solubility in water (comparative examples) was zero. Acceptance criteria: >3 times increase water solubility of the AFI/API in relation to its observed solubility

Additionally, a self-microemulsifying drug delivery system (SMEDDS) was prepared for Curcumin, where MCT oil was added under homogenization. A second dispersion was prepared of AvailOm® and water under homogenization and AvailOm® dispersion was added to MCT and API dispersion, which was kept under homogenization for further for 30-45 mins and spray dried afterwards (inlet temperature 70-73° C., Aspirator 92-100%). The spray dried material was then taken directly for analysis. Solubility of Curcumin in water was 8 μg/ml (>300% increase in solubility).

### Examples 8-13: Solubility Enhancement of API Celecoxib (Inventive)

TABLE 10
Formulation for API Celecoxib

| Experiment | I-8 | I-9 | I-10 | I-11 | I-12 | I-13 |
|---|---|---|---|---|---|---|
| Method of | PM | PM | PM | SD | SD | SD |
| Coprocessing | | | | | | |
| End Dosage | Tablet | Tablet | Tablet | Tablet | Tablet | Tablet |
| form | | | | | | |
| Ingredient | % w/w | % w/w | % w/w | % w/w | % w/w | % w/w |
| Celecoxib | 50 | 16.67 | 9.1 | 50.00 | 16.67 | 9.1 |
| AvailOm ® | 50 | 83.33 | 90.9 | 50.00 | 83.33 | 90.9 |
| Total | 100 | 100 | 100 | 100 | 100 | 100 |
| Ethanol: | .. | .. | .. | 70:30 | 70:30 | 70:30 |
| Water Ratio | | | | | | |
| Solid Content | .. | .. | .. | 37.5 | 40 | 52.38 |
| (% w/w) | | | | | | |

Formulations for the API Celecoxib are shown in table 10. For the physical mixing process (PM) applied for I-8 to I-10, AvailOm® was mixed in geometric addition by passing through a 30# sieve and the blend was directly taken for compression (parameters in table 11).

TABLE 11
Tableting parameters

| Parameter | I-8 | I-9 | I-10 |
|---|---|---|---|
| Punch size (mm) | 9 | 12.5 | 17.1*8.6 |
| Average Weight (mg) | 200 | 600 | 1100 |
| Compression force (KN) | 1.1612 | 5.77 | 8.269 |
| Ejection force (N) | 100.970 | 95 | 97.580 |
| Hardness (N) | 60-80 | 70-90 | 70-90 |

For the spray drying process (SD) applied for I-11 to I-13, Celecoxib was added in the non-aqueous media and to this solution AvailOm® was added under constant stirring. After addition of AvailOm®, the remaining amount of water was added and after the solution getting completely clear used for spray drying (table 12). The spray dried powder obtained was then mixed with other tableting excipients if any by passing through 30# sieve in geometric addition. The blend was taken for compression.

TABLE 12
Process parameter for spray drying

| Parameters | I-11 | I-12 | I-13 |
|---|---|---|---|
| Inlet Temperature (° C.) | 55-57 | 55 | 69 |
| Aspirator (%) | 98-100 | 90-97 | 90-100 |

TABLE 13
Tableting parameters

| Parameter | I-11 | I-12 | I-13 |
|---|---|---|---|
| Punch size (mm) | 9 | 12.5 | 17.1*8.6 |
| Average Weight (mg) | 200 | 600 | 1100 |
| Compression force (KN) | 1.1612 | 5.77 | 8.269 |
| Ejection force (N) | 100.970 | 95 | 97.580 |
| Hardness (N) | 60-80 | 70-90 | 70-90 |

The compressed tablets prepared according table 13 were analyzed in water to check whether there is any enhancement in solubility of the drug when compared to the pure form (table 14).

TABLE 14
Solubility of API in water,

| Experiment | I-8 | I-9 | I-10 | I-11 | I-12 | I-13 |
|---|---|---|---|---|---|---|
| Solubility of AFI in water | 2 μg/ml | 3 μg/ml | 26 μg/ml | 70 μg/ml | 291 μg/ml | 348 μg/ml |
| % Increase in Solubility | 2 × 10³ | 3 × 10³ | 26 × 10³ | 70 × 10³ | 29.1 × 10⁴ | 34.8 × 10⁴ |
| Acceptance criteria met | Yes | Yes | Yes | Yes | Yes | Yes |

Acceptance criteria: >3 times increase water solubility of the AFI/API in relation to its observed solubility

### Examples 14-24: Solubility Enhancement of API Ritonavir (Inventive)

TABLE 15
Formulation for API Ritonavir,

| Experiment | I-14 | I-15 | I-16 | I-17 | I-18 | I-19 | 1-20 | 1-21 | 1-22 | 1-23 | 1-24 |
|---|---|---|---|---|---|---|---|---|---|---|---|
| Method of | SD + PM | SD + PM | SD | SD + PM | SD + PM | PM | HME | Co- | Wet | Wet | SD |
| Coprocessing | | | | | | | | milling | granulat. | granulat. | |
| End Dosage form | Blend | Tablet | Blend | Blend | Blend | Blend | Blend | Blend | Blend | Blend | Blend |
| Ingredient | % w/w | % w/w | % w/w | % w/w | % w/w | % w/w | % w/w | % w/w | % w/w | % w/w | % w/w |
| Ritonavir | 10.21* | 10.21* | 14.78* | 14.20* | 11.9** | 37.04 | 18.2 | 18.2 | 18.2 | 25 | 20* |
| AvailOm ® | 24.50** | 24.50** | 22.17* | 8.53** | 17.83** | 29.63 | 27.3 | 27.3 | 27.3 | 75 | .. |
| EUDRAGIT ® E PO | 30.64 | 30.64 | 44.35* | 42.63* | 35.65* | .. | 54.54 | 54.54 | 54.54 | .. | .. |
| MCC PH 102 | 32.7 | 32.7 | 18.69 | 32.7 | 32.7 | 33.33 | .. | .. | .. | .. | 60* |
| Ac-di-sol | 1.96 | 1.96 | .. | 1.96 | 1.96 | .. | .. | .. | .. | .. | 10 |
| Total | 100 | 100 | 100 | 100 | 100 | 100 | .. | .. | .. | .. | 10 |
| Ethanol: Water Ratio | 100:0 | 100:0 | 90:10 | 100:0 | 100:0 | .. | 100 | 100 | 100 | 100 | 100 |
| Solid Content (% w/w) | 13.55 | 13.55 | 9.9 | 13.55 | 13.55 | .. | .. | .. | .. | .. | 90:10 |

*ingredients were co-processed together in a single spray drying step,
**ingredients were co-processed by only physically mixing with the spray dried powder in the same batch
Formulations for the API Ritonavir are shown in table 15.

For the spray drying process (SD) applied for I-16, the API was added in the non-aqueous media and EUDRAGIT® E PO was added, then AvailOm® was added under constant stirring to the mixture. After addition of AvailOm®, the remaining amount of water was added and after the solution getting completely clear used for spray drying (Inlet temperature: 55° C., Aspirator: 90-97%). The spray dried powder obtained was then mixed with other tableting excipients if any by passing through a 30# sieve in geometric addition. The blend was directly taken for analysis.

For the spray drying+physical mixing process (SD+PM) applied for I-14, I-15, I-17 and I-18, the API was added in the non-aqueous media and to this solution EUDRAGIT® E PO was added under constant stirring and after the solution getting completely clear used for spray drying (Inlet temperature: 42-43° C., Aspirator: 70-89). The spray dried powder obtained was then taken for physical mixing with AvailOm® and other tableting excipients (table 16). The blend (for I-14, I-17 and I-18) was directly taken for analysis.

TABLE 16
Tableting parameters

| Parameter | I-15 |
|---|---|
| Punch size (mm) | 12.5 |
| Average Weight (mg) | 490 |
| Compression force (KN) | 1.098 |
| Ejection force (N) | 108.650 |
| Hardness (N) | 70-90 |

For the physical mixing step applied for I-19, the API and AvailOm® were mixed with the other tableting excipients, then mixed in geometric addition by passing through a 30# sieve. The blend was directly taken for analysis.

For the hot melt extrusion (HME) step applied for I-20, all ingredients were mixed in geometric addition by passing through a 30# sieve. The blend was then directly taken for analysis.

For the co-milling process applied for I-21, all ingredients were mixed in geometric addition by passing through a 30# sieve. The blend was then co-milled in the mixer grinder by keeping the mixing interval of 3 mins. Then the co-milled blend was passed through a 50# sieve. The above process was repeated for three times. The final blend was directly used for analysis.

For the wet granulation process applied for I-22 and I-23, all ingredients were mixed in geometric addition by passing through a 30# sieve. The blend was then granulated with water in a planetary mixer, passed through a 12# sieve, then dried in a tray dryer for 30 mins and finally sifted through a 25# sieve. The final blend was directly used for analysis.

For the spray drying process applied for I-24, API was first added in ethanol under constant stirring, afterwards omega-3-fatty acid arginine salt was added to this solution, water was added and after the solution getting completely clear used for spray drying. The blend obtained was then mixed with the other tableting excipients and was directly used for analysis.

All the blends and tablets prepared according to table 15 were analyzed in water for any enhancement in solubility of the drug when compared to the pure form. The results are shown in table 17.

TABLE 17
Solubility of API in water,

| Experiment | I-14* | I-15* | I-16 | I-17 | I-18 | I-19 | I-20 | I-21 | I-22 | I-23 | I-24 |
|---|---|---|---|---|---|---|---|---|---|---|---|
| Solubility of | 80 | 70 | 140 | 20 | 30 | 40 | 208 | 76 | 40 | 239 | 272 |
| API in water | μg/ml | μg/ml | μg/ml | μg/ml | μg/ml | μg/ml | μg/ml | μg/ml | μg/ml | μg/ml | μg/ml |
| % Increase in | *>300% | *>300% | *>300% | *>300% | *>300% | *>300% | *>300% | *>300% | *>300% | *>300% | *>300% |
| Solubility | | | | | | | | | | | |
| Acceptance | Yes | Yes | Yes | Yes | Yes | Yes | Yes | Yes | Yes | Yes | Yes |
| criteria met | | | | | | | | | | | |

*The solubility in water (comparative examples) was zero. Acceptance criteria: >3 times increase water solubility of the AFI/API in relation to its observed solubility

### Comparative Examples 8-10

TABLE 18
Formulation for comparative experiments

| Experiment | C-8 | C-9 | C-10 |
|---|---|---|---|
| Method of Processing | . . . | . . . | Capsule filling |
| End Dosage form | API Powder | API Powder | Soft Gelatin Capsules |
| Ingredients | % w/w | % w/w | % w/w |
| Gliclazide | 100 | . . . | . . . |
| Carbamazepine | . . . | 100 | . . . |
| Cholecalciferol | . . . | . . . | 5.1 |
| Arachis Oil | . . . | . . . | 94.9 |
| Total | 100 | 100 | 100 |

For the comparative examples C-8 and C-9, the active ingredient was directly used for solubility analysis. For the example C-10, the active ingredient was diluted with carrier oil and was filled in soft gelatin capsules. The capsules were further analyzed for water solubility of the active ingredient. The results are summarized in table 19.

TABLE 19
Solubility of the active ingredients in water

| Experiment | C-8 | C-9 | C-10 |
|---|---|---|---|
| Solubility of active ingredient in water | 21 μg/ml | 240 μg/ml | 0 μg/ml |

### Examples 25-29: Solubility Enhancement of Gliclazide Using Different PUFA Salts (Inventive)

Formulations for Gliclazide with different salts of omega-3 fatty acids were prepared. For the spray drying process (SD) applied for I-25 to I-29, Gliclazide was added in alcohol and to this, an aqueous solution of omega-3-fatty acid salt was added under constant stirring. After the solution was completely clear, Aerosil® was added under stirring. The dispersion was spray dried using a spray drier (Inlet temperature: 55-60° C., Aspirator: 90-100%).

TABLE 20
Formulations for Gliclazide with different salts of omega-3-fatty acid

| Ingredient (% w/w) | I-25 | I-26 | I-27 | I-28 | I-29 |
|---|---|---|---|---|---|
| Gliclazide | 25 | 17.9 | 23.2 | 23.3 | 21.8 |
| Omega-3 lysine salt | 75 | . . . | . . . | . . . | . . . |
| Omega-3-fatty acid salt of (50%) | . . . | 53.6 | . . . | . . . | . . . |
| arginine & (50%) ornithine | | | | | |
| Omega-3-fatty acid salt of (50%) | . . . | . . . | 69.6 | . . . | . . . |
| ornithine & (50%) lysine | | | | | |
| Omega-3-fatty acid salt of (50%) | . . . | . . . | . . . | 69.76 | . . . |
| arginine & (50%) lysine | | | | | |
| Omega-3-fatty acid salt of (50%) | . . . | . . . | . . . | . . . | 65.22 |
| arginine & (50%) magnesium | | | | | |
| Aerosil ® | | 28.5 | 7.2 | 6.97 | 13.04 |
| Total | 100 | 100 | 100 | 100 | 100 |
| Solvent system (Ethanol:water) | 90:10 | 90:10 | 90:10 | 90:10 | 90:10 |
| Solid Content (% w/w) | 10 | 10 | 10 | 4 | 5 |

The spray dried powder was analyzed for solubility in water and was compared with solubility values from the comparative examples from table 18 (comparative example C-8). The data are summarized in table 21 below.

TABLE 21
Solubility of Gliclazide from its formulations. Acceptance
criteria: >3 times increase in solubility of the active ingredient
in formulation as compared to its solubility reported in table 18

| | I-25 | I-26 | I-27 | I-28 | I-29 |
|---|---|---|---|---|---|
| Solubility of Gliclazide in water (μg/ml) | 327 | 326 | 295 | 272 | 263 |
| % Increase in solubility | 1557 | 1552 | 1404 | 1295 | 1252 |
| Acceptance criteria met | Yes | Yes | Yes | Yes | Yes |

### Example 30: Solubility Enhancement of Carbamazepine (Inventive)

For the spray drying process (SD) applied for 1-30; Carbamazepine (9.1% w/w) was added in alcohol (ethanol:water ratio of 90:10) and an aqueous solution of the omega-3-fatty acid lysine salt (90.9 w/w) was added under constant stirring. The solution was spray dried using a spray drier (Inlet temperature: 55-60° C., Aspirator: 90-100%). The spray dried powder was analyzed for solubility in water and was compared with solubility values from the comparative examples from table 18 (comparative example C-9). The solubility of the API in water was 914 μg/ml, which corresponds to an increase in solubility of 381%, so the acceptance criteria (>3 times increase in solubility) was met.

### Examples 31-32: Solubility Enhancement of Vitamin D (Inventive)

For the SMEDDs in soft gel capsule applied for I-31 and I-32, the omega-3-lysine salt was sifted through #80 mesh and triturated with oil to form SMEDDs, size reduced to pass through #80 mesh and dispersed in carrier oil (summarized in table 22). This dispersion was filled in soft gelatin capsules and sealed.

TABLE 22
Formulations of Vitamin D3 with omega-3-lysine salt

| Ingredients (% w/w) | I-31 | I-32 |
|---|---|---|
| Vitamin D3 | 4.81 | 6.25 |
| Omega-3-lysine salt | 24.02 | 31.25 |
| Carrier Oil | Peanut Oil | Olive Oil |
| Carrier Oil % | 71.17 | 62.5 |
| Total | 100 | 100 |
| Vitamin D:Omega salt | 1:05 | 1:05 |

The soft gelatin capsules were analyzed for solubility in water and the values were compared with solubility values from the comparative examples from table 18 (comparative example C-10). The results are summarized in table 23.

TABLE 23
Solubility of Vitamin D3 in water, *The solubility in water
(comparative example C10) was zero. Acceptance criteria: >3 times
increase water solubility of the active ingredient in formulation as
compared to its solubility reported in table 19

| Ingredients (% w/w) | I-31 | I-32 |
|---|---|---|
| Solubility of Vitamin D3 in water (μg/ml) | 0.42 | 0.66 |
| % Increase in solubility | *More than | *More than |
| | 300% | 300% |
| Acceptance criteria met | Yes | Yes |

1. A method for enhancing the solubility of a pharmaceutical or nutraceutical active ingredient in an aqueous medium, the method comprising:
adding a preparation comprising at least one polyunsaturated fatty acid salt comprising at least one omega-3 fatty acid selected from the group consisting of eicosapentaenoic acid (EPA) and docosahexaenoic acid (DHA) to the medium comprising the pharmaceutical or nutraceutical active ingredient,
wherein the solubility is enhanced by at least 100% in comparison to the pharmaceutical or nutraceutical active ingredient in the aqueous medium alone.

2. The method of claim 1, wherein the at least one polyunsaturated fatty acid salt comprises at least one counter ion selected from the group consisting of lysine, arginine, ornithine, choline, magnesium, potassium, and mixtures thereof.

3. The method of claim 1, wherein the at least one polyunsaturated fatty acid salt comprises as a counter ion lysine, or
a mixture of lysine and one or more selected from the group consisting of arginine, ornithine, magnesium, and potassium,
wherein the ratio between the lysine and the arginine, ornithine, magnesium, and potassium is between 10:1 and 1:1.

4. The method of claim 1, wherein the preparation further comprises an ionic polymer.

5. A method of preparing a pharmaceutical or nutraceutical dosage form comprising at least one polyunsaturated fatty acid salt comprising at least one omega-3 fatty acid selected from EPA and DHA, and at least one pharmaceutical or nutraceutical active ingredient, the method comprising:
a. co-processing at least one polyunsaturated fatty acid salt comprising at least one omega-3 fatty acid selected from the group consisting of EPA and DHA, at least one pharmaceutical or nutraceutical active ingredient and optionally a pharmaceutically or nutraceutically acceptable excipient;
b. optionally mixing the co-processed components from step a. with one or more excipients; and
c. formulating the components to produce a dosage form.

6. The method of claim 5, wherein:
the dosage form is a tablet,
the mixed components from step b. are compressed in a tableting machine to produce a tablet, and
the ejection force experienced by the tableting machine is not more than 150 N.

7. The method of claim 5, wherein the ratio of the active ingredient to the polyunsaturated fatty acid salt is between 1:0.5 to 1:50.

8. The method of claim 5, wherein the polyunsaturated fatty acid salt and the pharmaceutical or nutraceutical active ingredient are co-processed with one or more ionic polymers.

9. The method of claim 8, wherein the ratio of the active ingredient to the ionic polymer is between 0.1:1 to 1:0.1.

10. The method of claim 8, wherein the ratio of polyunsaturated fatty acid salt and the ionic polymer is between 0.2:1 to 1:0.2.

11. The method of claim 5, wherein the co-processing in step a. comprises at least one selected from the group consisting of spray drying, pure spray drying, spray-agglomeration, co-milling, freeze drying, physical mixing, co-sifting, vacuum drying, hot-melt extrusion, compaction, slugging, 3D printing, molding, film casting, and coating.

12. The method of claim 5, wherein prior to step a. the pharmaceutical or nutraceutical active ingredient is mixed with one or more lipophilic substances.

13. The method of claim 5, wherein the pharmaceutical active ingredient is at least one selected from the group consisting of BCS classes II, III, and IV.

14. The method of claim 13, wherein the pharmaceutical active ingredient is ionic.

15. A pharmaceutical or nutraceutical dosage form prepared by the method of claim 5, wherein the dosage form is at least one selected from the group consisting of a tablet, a capsule, a soft capsule, a suspension, an emulsions, a granule, a powder, an oral film, a pellet, a suppository, a pessary, and an intra-vascular dosage form.

16. The pharmaceutical or nutraceutical dosage form of claim 15, further comprising at least one excipient selected from the group of consisting of a binder, an antioxidant, a glidant, a lubricant, a pigment, a plasticizer, a polymer, a brightener, a diluent, a flavor, a surfactant, a pore former, and a stabilizer.
